(19) United States
(12) Patent Application Publication
(10) Pub. No.: US 2015/0343216 A1
(43) Pub. Date: Dec. 3, 2015

(54) MODULATION OF ION CHANNELS

(71) Applicant: UNIVERSITY OF UTAH RESEARCH FOUNDATION, Salt Lake City, UT (US)

(72) Inventors: Steven Poelzing, Roanoke, VA (US); John Ryan Rigby, Magna, UT (US)

(21) Appl. No.: 14/648,549

(22) PCT Filed: Feb. 21, 2014
(86) PCT No.: PCT/US14/17544

§ 371 Date: May 29, 2015

Related U.S. Application Data
(60) Provisional application No. 61/768,271, filed on Feb. 22, 2013.

(51) Int. Cl. A61N 1/362

(52) U.S. Cl. CPC A61N 1/3628

(57) ABSTRACT

Electrical or magnetic fields oscillating at specific frequencies can modulate functions of ion channels in excitable cells. For example, an alternating current (AC) at a frequency that resonates with a particular type of ion channel can be applied to modulate conductance of the channel.

### CROSS-REFERENCE TO RELATED APPLICATIONS

This application claims priority to U.S. Provisional Patent Application No. 61/768,271 filed Feb. 22, 2013, which is incorporated herein by reference in its entirety.

### GOVERNMENT LICENSE RIGHTS

This invention was made with government support under grant number r21-h1094828 from the National Institutes of Health. The government has certain rights in the invention.

### BACKGROUND

In many living organisms, signals are transmitted between cells, such as neurons and muscle cells, by variations across cell membranes in electrophysiological parameters such as voltage, current or capacitance. Variations in such electrophysiological parameters often involve large numbers of multiple types of ion channels or receptors, which together produce a waveform at the biological cell. An action potential is an example of one type of waveform.

The waveform results from modulation of ion channels or receptors at the cell. For example, these ion channels or receptors may regulate the transmembrane and intercellular movement of physiological ions, such as Na⁺, K⁺, Ca²⁺, and Cl⁻, which form part of the signal. Modulation of one, or a group of ion channels or receptors results in electrophysiological changes at the membrane of the cell, causing further ion channels to be modulated. This process is closely coupled by feedback. Therefore the waveform produced at the biological cell varies depending on parameters such as the ion channels or receptors which are modulated and the length of time that those ion channels or receptors are activated or inhibited.

Ion channels have two fundamental properties, ion permeation and gating. Ion permeation describes the movement through the open channel. The selective permeability of ion channels to specific ions is a basis of classification of ion channels (e.g., Na⁺, K⁺, Ca²⁺ channels). Size, valency, and hydration energy are important determinants of selectivity. The selectivity ratio of the biologically important alkali cations is high. For example, the Na⁺:K⁺ selectivity of sodium channels is 10:1. Ion channels do not function as simple fluid-filled pores, but provide multiple binding sites for ions as they traverse the membrane. Ions become dehydrated as they cross the membrane as ion-binding site interaction is favored over ion-water interaction. Like an enzyme-substrate interaction, the binding of the permeating ion is saturable. Most ion channels are singly occupied during permeation; certain K⁺ channels may be multiply occupied. The equivalent circuit model of an ion channel is that of a resistor. The electrochemical potential, ΔV is the driving force for ion movement across the cell membrane. Simple resistors have a linear relationship between ΔV and current I (Ohm's Law, I=ΔV/R=ΔVg, where g is the channel conductance). Most ion channels have a nonlinear current-voltage relationship. For the same absolute value of ΔV, the magnitude of the current depends on the direction of ion movement into or out of the cells. This property is termed rectification and is an important property of K⁺ channels; they pass little outward current at positive (depolarized) potentials.

Gating is the mechanism of opening and closing of ion channels and is their second major property. Ion channels are also subclassified by their mechanism of gating: voltage-dependent, ligand-dependent, and mechano-sensitive gating. Voltage-gated ion channels change their conductance in response to variations in membrane potential. Voltage-dependent gating is the commonest mechanism of gating observed in ion channels. A majority of ion channels open in response to depolarization. The pacemaker current channel (I_{f} channel) opens in response to membrane hyperpolarization. The steepness of the voltage dependence of opening or activation varies between channels. Sodium channels increase their activation by ≈e-fold (2.73) for 4 mV of depolarization; in contrast, the K⁺ channel activation increase e-fold for 5 mV of depolarization.

Ion channels have two mechanism of closure. Certain channels like the Na⁺ and Ca²⁺ channels enters a closed inactivated state during maintained depolarization. To regain their ability to open, the channel must undergo a recovery process at hyperpolarized potentials. The inactivated state may also be accessed from the closed state. Inactivation is the basis for refractoriness in cardiac muscle and is fundamental for the prevention of premature re-excitation. The multiple mechanisms of inactivation are discussed below. If the membrane potential is abruptly returned to its hyperpolarized (resting) value while the channel is open, it closes by deactivation, a reversal of the normal activation process.

Arrhythmia is a variation from the normal rhythm of the heart beat. Cardiac arrhythmias are an important cause of morbidity and mortality. The major cause of fatalities due to cardiac arrhythmias is the subtype of ventricular arrhythmias known as ventricular fibrillation (VF). Conduction of electrical impulse is a unique property of cardiac and skeletal muscle and nervous tissue and is fundamental to their physiologic function. Abnormal cardiac electrical impulse generation and propagation underlies the pathogenesis of several diseases, including ventricular fibrillation.

Generally, three modes of therapies are used by the implantable defibrillators to treat dangerous arrythmias: 1) anti-tachycardia pacing; 2) low energy cardioversion; and 3) high energy defibrillation. Among the three, only high energy defibrillaton has been shown to be effective in defibrillating the heart during ventricular fibrillation.

There is a need for an improved device and method to treat arrhythmias and related heart diseases.

### SUMMARY

The subject technology generally relates to the use of electrical or magnetic fields oscillating at specific frequencies to modulate the functions of ion channels in excitable cells. In particular, an alternating current (AC) at a frequency that resonates with a particular type of ion channel is applied to modulate the function (such as conductance) of the ion channel.

In one aspect, the subject technology provides a method of modulating the conductance of an ion channel of an excitable cell, comprising: applying an alternating current (AC) at a frequency that resonates with said ion channel.

In another aspect, the subject technology provides a method of treating dysrhythmia in a subject in need thereof, comprising applying to said subject an alternating current (AC) at a frequency that resonates with an ion channel of a cardiomyocyte.

In another aspect, the subject technology provides a device for treating dysrhythmia, comprising: a computer or microprocessor-readable program containing one or more algorithms for generating or delivering alternating current (AC); a plurality of electrodes; and a waveform generator; wherein the device is configured to generate an alternating current (AC) at a frequency that resonates with an ion channel of a cardiomyocyte.

The subject technology is embodied by at least the following items:
1. A method of modulating the conductance of an ion channel of an excitable cell, comprising: applying an alternating current (AC) at a frequency that resonates with said ion channel.
2. The method of item 1, wherein said excitable cell is a cardiomyocyte.
3. The method of item 1 or 2, wherein said ion channel is a Na⁺ Channel.
4. The method of item 3, wherein said Na⁺ Channel is Naᵥ1.5
5. The method of item 3 or 4, wherein said frequency is about 25.5 kHz.
6. The method of item 3 or 4, wherein said frequency is about 40 kHz.
7. The method of item 1 or 2, wherein said ion channel is a K⁺ Channel.
8. The method of item 7, wherein said K⁺ Channel is Kᵢᵣ2.1
9. The method of item 7 or 8, wherein said frequency is about 31.8 kHz.
10. A method of treating dysrhythmia in a subject in need thereof, comprising applying to said subject an alternating current (AC) at a frequency that resonates with an ion channel of a cardiomyocyte.
11. The method of item 10, wherein said ion channel is a Na⁺ Channel.
12. The method of item 11, wherein said Na⁺ Channel is Naᵥ1.5
13. The method of item 10 or 11, wherein said frequency is about 25.5 kHz.
14. The method of item 10 or 11, wherein said frequency is about 40 kHz.
15. The method of item 10, wherein said ion channel is a K⁺ Channel.
16. The method of item 15, wherein said K⁺ Channel is Kᵢᵣ2.1
17. The method of item 15 or 16, wherein said frequency is about 31.8 kHz.
18. A device for treating dysrhythmia, comprising: a computer or microprocessor-readable program containing one or more algorithms for generating or delivering alternating current (AC); a plurality of electrodes; and a waveform generator; wherein the device is configured to generate an alternating current (AC) at a frequency that resonates with an ion channel of a cardiomyocyte.
19. The device of item 18, wherein said ion channel is a Na⁺ Channel.
20. The device of item 19, wherein said Na⁺ Channel is Naᵥ1.5
21. The device of item 18 or 19, wherein said frequency is about 25.5 kHz.
22. The device of item 18 or 19, wherein said frequency is about 40 kHz.
23. The device of item 18, wherein said ion channel is a K⁺ Channel.
24. The device of item 23, wherein said K⁺ Channel is Kᵢᵣ2.1
25. The method of item 23 or 24, wherein said frequency is about 31.8 kHz.

### BRIEF DESCRIPTION OF THE DRAWINGS

FIG. 1. Sodium Channel Correlation. The black line represents the correlation coefficient R as a function of wave frequency. The circles and bars represent the mean and standard deviation of the frequencies when R is 0, less or greater than 0.8 for all experiments.

FIG. 2. Potassium Channel Correlation. The gray line represents the correlation coefficient R as a function of wave frequency. The circles and bars represent the mean and standard deviation of the frequencies when R is 0, less or greater than 0.8 for all experiments.

FIG. 3. A 25.5 kHz, 5V sine wave significantly increases Transverse Conduction Velocity (cardiac conduction). *, p<0.05 relative to Pre and Post AC field stimulation.

FIG. 4. Currents through Nav1.5 at a step potential of −40 mV from a resting potential of −90 mV. Line 1 represents current due to a square step potential. Line 2 represents the current due to a step potential with a ±5 mV sinusoidal wave added to the step.

FIG. 5 depicts a cutaway drawing of an exemplary human heart showing the configuration of an embodiment of a dual chamber implantable cardiac pacemaker.

FIG. 6 is a block diagram illustrating an exemplary computer system 600 with which embodiments of the subject technology can be implemented.

### DETAILED DESCRIPTION

### A. Overview

The subject technology generally relates to the use of electrical or magnetic fields oscillating at specific frequencies to modulate the functions of ion channels in excitable cells. In particular, an alternating current (AC) at a frequency that resonates with a particular type of ion channel is applied to modulate the function (such as the conductance) of the ion channel.

Current cardiac electropharmacotherapy relies on pacing or applying large potentially damaging electrical stimuli to tissue in order to bluntly reset or resynchronize electrical activity. State-of-the Art technologies rely on indiscriminately activating or inhibiting all cells within a region without specificity to particular protein function. The application of electro-pharmacotherapy seeks to grossly reset systems or entrain systems, such that when the electro-pharmacotherapy is released, excitable cells can presumably return to some resting state ready for some normal electrical excitatory pathway.

There are two main limitations with this approach. The first has to do with the timing of the therapy. Electro-pharmacotherapy can be applied as the substrate for arrhythmias are developing and by modulating the pacing rates (anti-tachycardia pacing, ATP), with the end point of preventing the arrhythmia. Alternatively, electropharmacotherapy is employed after abnormal electrical activity has already begun which generally is associated with a loss of patient consciousness and significantly altered neuro-humoral cardiac responses. ATP paradoxically achieves its efficacy by increasing the heart rate in an adaptive manner, a condition associated with accumulation of intracellular calcium and increased propensity for calcium mediated triggered activity in disease. The second issue is that these devices are grossly and very indirectly addressing the individual abnormalities. Specifically, if cardiac alternans of action potential duration (APD) are linked to arrhythmogenicity, ATP pacing could be used to adjust APD, but the underlying cause of APD alternans are thought to be calcium mediated. Therefore, a more direct approach with lower power requirements might be to specifically modulate ion channels responsible for intracellular calcium accumulation in real-time.

Rigby et al. (Ann Biomed Eng. 2012 April; 40(4):946-54) discloses that ion channels may exhibit unique frequency responses, and further discloses methods for relating ion channel frequency response with channel conductance. However, Rigby does not demonstrate or suggest that the oscillating field affects channel conductance. The subject technology discloses that when there exits a correlation between which frequencies are preferentially passed or damped by an ion channel, oscillating fields at specific frequencies can be applied to modulate the behavior of specific ion channels.

WO 2011/029029 discloses methods and devices for termination of arrhythmias, such as ventricular or atrial tachyarrhythmias. The device and method involves application of alternating current (AC) for clinically significant durations at selected therapeutic frequencies through the cardiac tissue to a subject experiencing arrhythmia. Methods are also provided to minimize or eliminate pain during defibrillation. However, the preferred frequencies of AC are said to be between about 50 Hz to 500 Hz. Correlation between a frequency and ion channel function has not been disclosed.

The subject technology is based in part on the discovery that, by applying a sinusoidal electric field across the heart at a frequency that resonates with the cardiac sodium channel Nav1.5, the inventors were able to increase cardiac conduction, consistent with a therapy that specifically targets cardiac sodium channels.

Accordingly, in one aspect, the subject technology provides a method for modulating the conductance of an ion channel of an excitable cell, comprising: applying an alternating current (AC) at a frequency that resonates with said ion channel.

In another aspect, the subject technology provides a method of treating dysrhythmias (such as cardiac fibrillation or tachycardia) in a subject in need thereof, comprising applying to said subject an alternating current (AC) at a frequency that resonates with an ion channel of a cardiomyocyte.

Also described herein are devices for treating dysrhythmias (such as cardiac fibrillation or tachycardia).

### B. Definitions

As used herein, the singular forms “a,” “an” and “the” include plural references unless the content clearly dictates otherwise.

The term “about”, as used here, refers to +/−10% of a value.

The frequency of an alternating current “resonates” with an ion channel when correlation coefficient (R) meets the criterion: |R| is greater than 0.5, preferably greater than 0.6, more preferably greater than 0.7, and even more preferably greater than 0.8. Specifically, frequencies with absolute correlation coefficients greater than 0.8 represent frequencies whose magnitudes can be used to recreate the DC component of the ionic current. Similarly, frequencies with correlation coefficients equal to zero (termed ‘zero crossings’) represent frequencies whose magnitudes are independent of the ionic current.

The terms “treating” and “treatment” refer to alleviating, inhibiting, or reversing the progress of the disorder or condition, or one or more symptoms associated with such disorder or condition. The terms also encompass prophylactic treatment or prevention.

### C. Modulating the Functions of Ion Channels

1. Ion Channels

Ion channels are pore-forming membrane proteins whose functions include establishing a resting membrane potential, shaping action potentials and other electrical signals by gating the flow of ions across the cell membrane, controlling the flow of ions across secretory and epithelial cells, and regulating cell volume. Ion channels are present in the membranes of all cells. Ion channels are considered to be one of the two traditional classes of ionophoric proteins, with the other class known as ion transporters (including the sodium-potassium pump, sodium-calcium exchanger, and sodium-glucose transport proteins, amongst others).

Sodium channels are integral membrane proteins that form ion channels, conducting sodium ions (Na+) through a cell's plasma membrane. They are classified according to the trigger that opens the channel for such ions, i.e. either a voltage-change (“Voltage gated”, “voltage sensitive” or “voltage dependant” sodium channel also called “VGSCs” or “Nav channel”) or binding of a substance (a ligand) to the channel (ligand-gated sodium channels).

Voltage-gated sodium channels normally comprise an alpha subunit that forms the ion conduction pore and one to two beta subunits that have several functions including modulation of channel gating. Expression of the alpha subunit alone is sufficient to produce a functional channel. The family of sodium channels has nine known members, with amino acid identity >50% in the trans-membrane segments and extracellular loop regions. A standardized nomenclature for sodium channels is currently used and is maintained by the IUPHAR.

TABLE 1
Nomenclature and some functions of voltage-gated sodium channel alpha subunits

| Protein | | | |
|---|---|---|---|
| name | Gene | Expression profile | Associated human channelopathies |
| Naᵥ1.1 | SCN1A | Central neurons, | febrile epilepsy, GEFS+, Dravet syndrome (also known as severe |
| | | [peripheral neurons] | myclonic epilepsy of infancy or SMEI), borderline SMEI (SMEB), |
| | | and cardiac myocytes | West syndrome (also known as infantile spasms), Doose syndrome |
| | | | (also known as myoclonic astatic epilepsy), intractable childhood |
| | | | epilepsy with generalized tonic-clonic seizures (ICEGTC), |
| | | | Panayiotopoulos syndrome, familial hemiplegic migraine (FHM), |
| | | | familial autism, Rasmussens's encephalitis and Lennox-Gastaut |
| | | | syndrome |
| Naᵥ1.2 | SCN2A | Central neurons, | inherited febrile seizures and epilepsy |
| | | peripheral neurons | |
| Naᵥ1.3 | SCN3A | Central neurons, | none known |
| | | peripheral neurons and | |
| | | cardiac myocytes | |
| Naᵥ1.4 | SCN4A | Skeletal muscle | hyperkalemic periodic paralysis, paramyotonia congenita, |
| | | | and potassium-aggravated myotonia |
| Naᵥ1.5 | SCN5A | Cardiac myocytes, | Long QT syndrome, Brugada syndrome, and idiopathic ventricular |
| | | uninnervated skeletal | fibrillation |
| | | muscle, central neurons | |
| Naᵥ1.6 | SCN8A | Central neurons, dorsal | none known |
| | | root ganglia, peripheral | |
| | | neurons, heart, glia | |
| | | cells | |
| Naᵥ1.7 | SCN9A | Dorsal root ganglia, | erythromelalgia, PEPD, channelopathy-associated insensitivity to |
| | | sympathetic | pain and recently discovered a disabling form |
| | | neurons, Schwann cells, | offibromyalgia (rs6754031 polymorphism - PMID 22348792). |
| | | andneuroendocrine | |
| | | cells | |
| Naᵥ1.8 | SCN10A | Dorsal root ganglia | none known |
| Naᵥ1.9 | SCN11A | Dorsal root ganglia | none known |
| Naₓ | SCN7A | heart, uterus, skeletal | none known |
| | | muscle, astrocytes, | |
| | | dorsal root ganglion | |
| | | cells | |

TABLE 2
Nomenclature and some functions of voltage-
gated sodium channel beta subunits

| | | | | Associated |
|---|---|---|---|---|
| | | | | human |
| Protein | Gene | | | channel- |
| name | link | Assembles with | Expression profile | opathies |
| Naᵥβ1 | SCN1B | Naᵥ1.1 to Naᵥ1.7 | Central Neurons, | epilepsy |
| | | | Peripheral Neurons, | (GEFS+) |
| | | | skeletal muscle, | |
| | | | heart, glia | |
| Naᵥβ2 | SCN2B | Naᵥ1.1, Naᵥ1.2, | Central Neurons, | none known |
| | | Naᵥ1.5 to Naᵥ1.7 | peripheral neurons, | |
| | | | heart, glia | |
| Naᵥβ3 | SCN3B | Naᵥ1.1 to Naᵥ1.3, | central neurons, | none known |
| | | Naᵥ1.5 | adrenal gland, | |
| | | | kidney, peripheral | |
| | | | neurons | |
| Naᵥβ4 | SCN4B | Naᵥ1.1, Naᵥ1.2, | heart, skeletal | none known |
| | | Naᵥ1.5 | muscle, central and | |
| | | | peripheral neurons | |

There are four major classes of potassium channels: Calcium-activated potassium channel, Inwardly rectifying potassium channel, Tandem pore domain potassium channel, and Voltage-gated potassium channel, as shown in Table 3.

TABLE 3
Potassium channel classes, function, and pharmacology.

| Class | Subclasses | Function | Blockers | Activators |
|---|---|---|---|---|
| Calcium- | BK channel | inhibition following stimuli | charybdotoxin, iberiotoxin | 1-EBIO |
| activated | SK channel | increasing intracellular | apamin | NS309 |
| 6T & 1P | | calcium | | CyPPA |
| Inwardly | ROMK (Kᵢᵣ1.1) | recycling and secretion of | Nonselective: Ba²⁺, Cs⁺ | none |
| rectifying | | potassium in nephrons | | |
| 2T & 1P | GPCR regulated (Kᵢᵣ3.x) | mediate the inhibitory | GPCR antagonists | GPCR agonists |
| | | effect of many GPCRs | ifenprodil | |
| | ATP-sensitive (Kᵢᵣ6.x) | close when ATP is high to | glibenclamide | diazoxide |
| | | promote insulin secretion | tolbutamide | pinacidil |
| Tandem | TWIK (TWIK-1, TWIK- | Contribute to resting | bupivacaine | halothane |
| pore | 2, KCNK7) | potential | quinidine | |
| domain | TREK(TREK-1, TREK- | | | |
| 4T & 2P | 2, TRAAK) | | | |
| | TASK(TASK-1, TASK- | | | |
| | 3, TASK-5) | | | |
| | TALK (TASK-2, TALK- | | | |
| | 1, TALK-2) | | | |
| | THIK (THIK-1, THIK-2) | | | |
| | TRESK | | | |
| Voltage- | hERG (Kᵥ11.1) | action | tetraethylammonium | retigabine (Kᵥ7) |
| gated | KvLQT1 (Kᵥ7.1) | potential repolarization | 4-aminopyridine | |
| 6T & 1P | | limits frequency of action | dendrotoxins (some | |
| | | potentials (disturbances | types) | |
| | | cause dysrhythmia) | | |

A Calcium channel is an ion channel which displays selective permeability to calcium ions. Voltage-dependent calcium channels (VDCC) are a group of voltage-gated ion channels found in excitable cells (e.g., muscle, glial cells, neurons, etc.) with a permeability to the ion Ca2+. These channels are slightly permeable to sodium ions, so they are also called Ca2+-Na+ channels, but their permeability to calcium is about 1000-fold greater than to sodium under normal physiological conditions. At physiologic or resting membrane potential, VDCCs are normally closed. They are activated (i.e., opened) at depolarized membrane potentials and this is the source of the “voltage-dependent” epithet. Activation of particular VDCCs allows Ca2+ entry into the cell, which, depending on the cell type, results in muscular contraction, excitation of neurons, up-regulation of gene expression, or release of hormones or neurotransmitters.

Voltage-dependent calcium channels are formed as a complex of several different subunits: α₁, α₂δ, β₁₋₄, and γ. The α₁ subunit forms the ion conducting pore while the associated subunits have several functions including modulation of gating.

TABLE 4
Calcium channels

| Type | Gated by | Protein | Gene | Location | Function |
|---|---|---|---|---|---|
| L-type | high voltage | Caᵥ1.1 | CACNA1S | Skeletal muscle, bone | SMC and cardiac muscle contraction. |
| | | Caᵥ1.2 | CACNA1C | (osteoblasts), ventricular | Responsible for prolonged action |
| | | Caᵥ1.3 | CACNA1D | myocytes, dendrites and | potential in cardiac muscle. |
| | | Caᵥ1.4 | CACNA1F | dendritic spines of cortical | |
| | | | | neurons | |
| P-type/ | high voltage | Caᵥ2.1 | CACNA1A | Purkinje neurons in the | neurotransmitter release |
| Q-type | | | | cerebellum/ | |
| | | | | Cerebellar granule cells | |
| N-type | high voltage | Caᵥ2.2 | CACNA1B | Throughout the brain | neurotransmitter release |
| R-type | intermediate | Caᵥ2.3 | CACNA1E | Cerebellar granule cells, | |
| | voltage | | | other neurons | |
| T-type | low voltage | Caᵥ3.1 | CACNA1G | neurons, cells that have | Regular sinus rhythm |
| | | Caᵥ3.2 | CACNA1H | pacemaker activity, bone | |
| | | Caᵥ3.3 | CACNA1I | (osteocytes) | |

Chloride channels are a superfamily of poorly understood ion channels having approximately 13 members. Chloride channels are important for setting cell resting membrane potential and maintaining proper cell volume.

2. Frequency Analysis

Frequencies that resonate with a particular ion channel can be determined using the methods as described in Rigby J R, Poelzing S. Recreation of an Ion Channel Current IV Curve Using Frequency Components. J Vis Exp. 2011 Feb. 8; (48), and Rigby J R, Poelzing S. A Novel Frequency Analysis Method for Assessing K(ir)2.1 and Na (v)1.5 Currents. Ann Biomed Eng. 2012 April; 40(4):946-54.

For example, Rigby (2012) describes a technique that involves inserting a noise function into a standard voltage step protocol, which allows one to characterize the unique frequency response of an ion channel at different step potentials. Specifically, the fast Fourier transform is computed for a family of current traces at different step potentials for the inward rectifying potassium channel, Kir2.1, and the channel encoding the cardiac fast sodium current, Nav1.5. Each individual frequency magnitude, as a function of voltage step, is correlated to the peak current produced by each channel. The correlation coefficient vs. frequency relationship reveals that these two channels are associated with some unique frequencies with high absolute correlation. The individual IV relationship can then be recreated using only the unique frequencies with magnitudes of high absolute correlation. Thus, using the methods described by Rigby (2012), unique resonant frequencies that trigger ion channels responses may be ascertained.

As exemplified herein, it has been discovered that 8.3 kHz fields do not correlate with Nav1.5 conductance, but positively correlate with Kir2.1 conductance; 25.5 kHz fields positively correlate with Nav1.5 conductance, but not Kir2.1 conductance; 31.8 kHz fields do not correlate with either Nav1.5 or Kir2.1 conductance; 40 kHz fields demonstrate weak inverse correlation with Nav1.5 but no correlation with Kir2.1.

3. Methods and Devices for Modulating Ion Channel Functions

Accordingly, in one aspect, the subject technology provides a method for modulating the conductance of an ion channel of an excitable cell, comprising: applying an alternating current (AC) at a frequency that resonates with said ion channel.

In another aspect, the subject technology provides a method of treating dysrhythmias (such as cardiac fibrillation or tachycardia) in a subject in need thereof, comprising applying to said subject an alternating current (AC) at a frequency that resonates with an ion channel of a cardiomyocyte.

Alternating current may be delivered in any number of waveforms or combinations or waveforms. In various embodiments, the waveform may be a sinusoidal, triangular, or square-wave, as well as any combinations thereof. Additionally, square-waves, may have a duty-cycle of about 25%, 30%, 35%, 40%, 45%, 50%, 55%, 60%, 65%, 70%, or 75%. Further, the waveform may switch on or off abruptly, or may be shaped by an envelope waveform to effect more gradual onset or offset.

Alternating current may be applied or administered for various durations of time ranging from about 0.025 second to 2 seconds to accomplish termination of the arrhythmia. In various embodiments, alternating current may be applied or administered for about 0.025 second to 1.5 seconds, or 0.025 second to 1 second, 0.025 to 0.5 second. For example, alternating current may be applied or administered for about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2.0 seconds.

The subject technology also provides devices for treating dysrhythmias (or arrhythmias), such as cardiac fibrillation or tachycardia. The device may be accompanied by instructions instructing health care providers or patients suitable frequencies to be applied. The device may be configured to apply alternating current manually at the discretion of a health care worker, either by an internally implanted or externally applied device, or may be applied automatically in response to a detected arrhythmia, either by an implanted or externally disposed device. Such applications may coincide with detection of arrhythmia in the subject by a sensing circuit allowing detection of the arrhythmia, which may be included in or external to the device.

For example, devices described herein can be similar to cardiac defibrillators, which are medical devices for treating patients who have experienced an episode of ventricular tachycardia or ventricular fibrillation. Cardiac defibrillators are often implanted within a patient to detect and treat ventricular tachycardia or ventricular fibrillation. Implantable cardioverter-defibrillators (ICDs) include a small battery-powered electrical impulse generator that is implanted in patients who are at risk of ventricular fibrillation. The ICDs are programmed to detect cardiac arrhythmia and correct it by delivering a jolt of electricity through electrodes that are introduced into the heart. Devices that deliver ACs of resonant frequencies, as described herein, can function similar to that of cardiac defibrillators.

The device and methods may be used to treat a number of different types of dysrhythmias. Cardiac arrhythmia, also known as “dysrhythmia,” is a rubric for a group of conditions characterized by abnormal electrical activity in the heart. Examples of arrhythmias include premature ventricular contractions, ventricular tachycardia, ventricular fibrillation and supraventricular tachyarrhythmia such as atrial fibrillation. By example, atrial fibrillation (AF) is a supraventricular tachyarrhythmia characterized by uncoordinated atrial activation with consequent deterioration of atrial mechanical function. Persistent and/or chronic AF is associated with increased risk of thromoembolic events including MI and stroke and heart failure. Theories of the mechanism of AF involve two main processes: enhanced automaticity in one or several rapidly depolarizing foci and reentry involving one or more circuits.

In certain embodiments, the dysrhythmia is a tachyarrhythmia, such as ventricular tachyarrhythmia, or atrial tachyarrhythmia Ventricular tachyarrhythmias may include, but are not limited to ventricular fibrillation. Atrial tachyarrhythmias may include, but are not limited to atrial fibrillation and atrial flutter.

The methods and devices described herein may also be used to treat other conditions associated with inappropriate ion channel activities. For example, existing sodium channel blockers have been used to treat a number of diseases, including epilepsy, bipolar disease, depression, pain, ALS, neurodegenerative diseases and arrhythmia.

The device and methods may utilize a plurality of electrodes which may be configured in a variety of ways to administer alternating current. Alternating current may be administered via a number of electrode configurations as described. When used with an externally applied device, the alternating current is preferably applied via large electrodes placed on the skin across the heart as is typically done with external defibrillators. Automatic response to a cardiac event (such as arrhythmia) detection can be implemented using separate skin electrodes to detect the ECG, or using the same large electrodes through which the alternating current is then applied.

When used with an implanted device, the alternating current may be applied via electrodes placed in or about the cardiac chambers, or via electrodes placed in the chest outside the rib cage, for example in the subcutaneous layers including the housing of the implanted device itself, or using a combination of such electrodes. Automatic response to arrhythmia detection can be accomplished using electrodes placed in or about the cardiac chamber or chambers susceptible to tachyarrhythmia, or using electrodes placed in the chest outside the ribcage, for example in the subcutaneous layers.

In one configuration, a device may be in electrical communication with a subject's heart by way of one or more leads, suitable for delivering multi-chamber stimulation and pacing therapy. Not every configuration has all of the electrodes to be described below, but a particular configuration may include some of these electrodes. Other configurations of the device may include even more electrodes than discussed herein. For example, alternating current may be applied by other, additional electrodes than those described below. Further, the electrodes and device may be configured to apply alternating current using a tiered approach. Additional electrodes for delivering alternating current can include combinations or electrodes situated over the epicardium (e.g., multiple pacing and relatively larger surface area defibrillation electrodes that may be used for optimizing cardiac resynchronization therapy and providing defibrillation).

Regarding the leads and electrodes, in order to sense atrial cardiac signals and to provide right atrial chamber stimulation therapy, the device may be coupled to an implantable right atrial lead, typically having an atrial tip electrode and an atrial ring electrode, which may be implanted in the subject's right atrial appendage. The device is also known as and referred to as a pacing device, a pacing apparatus, a cardiac rhythm management device, or an implantable cardiac stimulation device.

To sense left atrial and ventricular cardiac signals and to provide left atrial and ventricular pacing therapy, the device may be coupled to a “coronary sinus” lead configured for placement in the “coronary sinus region” via the coronary sinus opening for positioning a distal electrode adjacent to the left ventricle or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, a coronary sinus lead may be configured to receive atrial and ventricular cardiac signals and to deliver left ventricular pacing therapy using a left ventricular (LV) tip electrode and a LV ring electrode. Left atrial pacing therapy may use, for example, first and second left atrial (LA) ring electrodes. Administration of alternating current can also be performed using at least a coronary sinus coil electrode. Administration of alternating current can also be performed using a pair of right atrial (RA) ring electrodes.

The device may also be in electrical communication with a subject's heart by way of an implantable right ventricular lead, typically having a right ventricular (RV) tip electrode, an RV ring electrode, an RV coil electrode, and a superior vena cava (SVC) coil electrode (also known as a right atrial (RA) coil electrode).

The components of the device may be contained in a case, which is often referred to as the “can”, “housing”, “encasing”, or “case electrode”, and may be programmably selected to act as the return electrode for unipolar operational modes. The case may further be used as a return electrode alone or in combination with one or more additional electrodes for stimulating purposes. The case may further include a connector having a plurality of terminals for connecting one or more of the following electrodes in various configurations:

a left ventricular tip electrode;

a left ventricular ring electrode;

a left atrial coil electrode;

a left atrial ring electrode(s);

a coronary sinus coil electrode;

a right ventricular tip electrode;

a right ventricular ring electrode;

a right ventricular RV coil electrode;

right atrial ring electrode(s); a right atrial tip electrode;

a right atrial SVC coil electrode;

an epicardial electrode; and subcutaneous electrode(s).

The device and methods described herein include tiered therapy, which provides an adaptive and refined therapy for a cardiac event (such as arrhythmia). The tiered approach divides therapy for arrhythmias into a progression of multiple tiers. For example, tiered therapy may include applying alternating current along a progression of different durations, the progression continuing until the cardiac event (such as arrhythmias) is terminated.

The subject technology described herein also encompass prevention (prophylactic treatment) of dysrhythmias (or arrhythmias), cardiac fibrillation, or tachycardia. AC at resonant frequencies can be applied to prevent development of pro-arrhythmic states.

Patients at risk of, or suffering from dysrhythmia can be assessed or monitored according to the standard practice in the art. For example, a device for detecting a cardiac event, including, for example, sensing cardiac signals from a plurality of electrodes, determining rates of change of the sensed cardiac signals, or determining a range of the sensed cardiac signals, may be used. The sensed cardiac signals may be associated with a cardiac event that needs treatment. Further determination may be made to associate the functions of a particular type ion channels with the cardiac event, and the resonant frequencies of the ion channels that can change the conductance of the ion channel. A computer-implemented system for assessing a patient's risk for dysrhythmia may be used. Devices assessing or monitoring a patient's condition may be used together with a treatment device that delivers AC of resonant frequencies. For example, monitoring devices may be used after treatment to determine the efficacy of the treatment, and to determine whether certain adjustment needs to be made.

To analyze the heart's operation, a variety of techniques have been developed for collecting and interpreting data concerning the electrical activity of the heart. One of the most basic of these approaches is the electrocardiogram (ECG). As an electrical signal spreads across the heart, an ECG repetitively measures the voltages at various electrodes relative to a designated “ground” electrode. The ECG typically plots each lead over an interval of time such that the heart's electrical activity for one or more cardiac cycles is displayed for purposes of monitoring or analysis. The three most common ECG's are known as the “12 lead”, the “18 lead,” and the vector cardiograph.

A cardiac cycle as measured by the ECG is partitioned into three main elements which reflect the electrical and mechanical operation of the heart. The portion of a cardiac cycle representing atrial depolarization is referred to as a “P-wave.” Depolarization of the ventricular muscle fibers is represented by “Q”, “R”, and “S” points of a cardiac cycle. Collectively these “QRS” points are called an “R-wave” or a “QRS complex.” The portion of a cardiac cycle representing repolarization of the ventricular muscle fibers is known as a “T-wave.” It is through the use of an ECG that one is able to determine whether fibrillation is or is not occurring and allows one to manipulate the heart tissue to provide treatment.

In certain embodiments, the device can be part of a pacemaker, or used in connection/conjunction with a pacemaker. A pacemaker maintains the heart rate of a patient between a certain programmable range. For example, in humans that range is typically between 60-80 beats per minute (lower rate) and 120-160 beats per minute (upper rate). A pacemaker automatically applies a pacing impulse to the heart of sufficient magnitude to depolarize the tissue. The device is adapted to continue delivering intermittent pacing to the heart in the event that the heart fails to return to its normal behavioral pattern, and has the ability of automatically regaining sensing control over a functional heart, thereby insuring that further pacing is inhibited.

The pacemaker circuit comprises two basic subsystems; a sensing system, which continuously monitors heart activity; and a stimulation system which upon receiving a signal from the sensing system applies a pacing impulse to the myocardium through an intravascular electrical lead. A first bipolar lead may be coupled to the pulse generator and has an electrode located at its distal end to sense and pace the atrium. Alternatively, the atrial leads may comprise separate sensing and pacing electrodes. A second bipolar lead coupled to the generator is used for sensing and pacing the ventricle. Alternatively, the ventricular leads may comprise separate sensing and pacing electrodes. A circuit is provided for applying impedance measuring current pulses between one of these electrodes and the others.

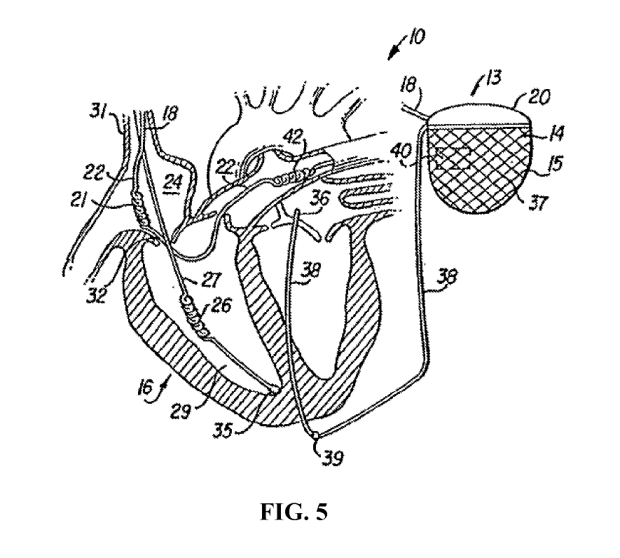

For example, FIG. 5 provides one exemplary embodiment. The pacemaker is implanted in a surgically-formed pocket in the flesh of the patient's chest 10, or other desired location of the body. Signal generator 14 is conventional and incorporates electronic components for performing signal analysis and processing, waveform generation, data storage, control and other functions, power supply (battery or battery pack), which are housed in a metal case (can) 15 compatible with the tissue and fluids of the body (i.e., biocompatible). The device is microprocessor-based with substantial memory, logic and other components to provide the processing, evaluation and other functions necessary to determine, select and deliver appropriate therapy, such as delivering pulses of different energy levels, frequencies, or timing for treating cardiac fibrillation, for cardioversion, or for pacing at the patient's heart 16.

Composite electrical lead 18 which includes separate leads 22 and 27 with distally located electrodes is coupled at the proximal end to signal generator 14 through an electrical connector 20 in the header of case 15. Preferably, case 15 is also employed as an electrode such as electrical ground, for unipolar sensing, pacing (or defibrillation if needed). The signal generator and lead(s) of the present invention may be implemented for atrial and ventricular sensing, pacing and defibrillation. Electrical currents of appropriate energy level and frequency may be applied between the case and electrode 21 on lead 22 implanted in the right atrium 24 through the superior vena cava 31, or between the case and electrode 26 on lead 27 implanted through the superior vena cava in the right ventricle 29. Leads 22 and 27 and their associated distal tip electrode 32 (to a separate conductor) and distal tip electrode 35 (also to a separate conductor within the lead), respectively, may be used for both a sensing lead and a pacing lead in conjunction with the circuitry of signal generator 14. One of skill in the art may easily recognize that separate sensing and pacing leads are also compatible with this described system. To that end, electrode 32 is positioned in the right atrium against either the lateral or anterior atrial wall thereof, and electrode 35 is positioned in the right ventricle at the apex thereof

Active or passive fixation of the electrodes may be used to assure suitable excitation. Tip electrode tip 35 preferably has a standard 4 to 8 millimeter (mm) configuration, and is provided with soft barbs (tines) to stabilize its position in the ventricle. Each of the electrodes, those used for defibrillation and cardioversion, as well as those used for sensing and for pacing, are electrically connected to separate conductors in leads 22 and 27.

If desired, rather than simply using metal case 15 as an electrode, a conductive pouch 37 comprised of a braided multiplicity of carbon fine, individual, predominantly isotropic wires, such as described in U.S. Pat. No. 5,143,089, is configured to receive, partly enclose and maintain firm electrical contact with the case. The conductive pouch can be electrically connected directly to an extension lead 38 composed of similar carbon braid of about 7 french diameter which is implanted subcutaneously for connection to an epicardial or pericardial patch electrode (not shown) or as a wire electrode (as shown) through an opening formed by puncture surgery at 39. The conductor for electrode 36 of lead 38 may be implanted subcutaneously to a point 39, and then by puncture surgery through the thoracic cage and the pericardial sac, under a local anesthetic. The lead 38 is run parallel to the sternum, through the puncture, and then through the patient's thoracic cage and into the pericardial sac. It may even be threaded through the thoracic cage, the pericardial space about the left ventricle and atrium, and back along the right atrial appendage, external to the heart. The distal end 36 of lead 38 may be placed close to the left atrium of the patient's heart to provide an increase in electric field strength and support the strong vector of the electric field according to the heart chamber to be defibrillated. Selection of the chamber (i.e., atrium or ventricle) which is to undergo defibrillation is made by choosing the appropriate endocardial counter-electrode (21 or 26, respectively) to be energized together with the carbon electrode, if the case 15 or conductive pouch 37 is not used directly as the other electrode.

The electrode portion of conductor 38 (from the point of entry 39 into the thoracic cage) may be made with carbon braid.

Atrial coil electrode 21 may be used for bipolar sensing as well as a counter-electrode for defibrillation. Ventricular electrode 26 of lead 27 is positioned for use as a defibrillation electrode as well as for bipolar sensing in the ventricle. For defibrillation, electrode 26 also cooperates with the metal case 15, pouch electrode 37, or pericardial electrode 36, whichever of these latter electrodes is used in the defibrillator implementation. The electrode may be composed of carbon braid, fine metallic filaments and fibers of platinum iridium alloy, braided together to offer similarly desirable electrode characteristics.

The tip electrodes of leads 22 and 27 may be used for sensing and pacing of the respective atrial and ventricular chambers as in a conventional pacemaker, with dual-chamber pacing, dual-chamber sensing, and both triggered and inhibited response. Further, the defibrillator 13 uses a transvenous electrode for ventricular defibrillation and stimulation and an atrial bipolar lead for sensing and atrial defibrillation, so that atrial defibrillation is performed with one of the same electrodes used for atrial stimulation and sensing.

Rather than terminating at distal tip electrode 32, the latter electrode may be positioned at mid-lead of the atrial transvenous lead 22 which extends and is threaded through right atrium, ventricle, pulmonary valve, and into the left pulmonary artery, with a coil counter-electrode 42 connected to a separate conductor of the lead. With this alternative embodiment, a defibrillating waveform can be applied between electrode 42 and atrial defibrillation electrode 21 upon detection of atrial fibrillation. In that configuration, electrode 42 would replace signal generator case 15, conductive pouch 37, or lead portion 36 as the selected electrode, and enables a strong vector for the electric field through right and left atrium. Rather than placement in the left pulmonary artery, electrode 42 may be positioned in the distal coronary sinus for defibrillation of the atrium in conjunction with electrode 21.

Defibrillation of the atrium and ventricle is achieved by application of defibrillation waveforms of suitable shape and energy content between appropriate electrodes, such as electrode 36 and electrode 21 for atrial fibrillation, or between electrode 42 and electrode 21 for atrial fibrillation; or between electrode 36 and electrode 26 for ventricular fibrillation, in, which atrial electrode 21 can be used additionally as either anode or cathode. The case 15 can serve as the anode for delivery of the shock as well, and can provide ground reference potential for unipolar sensing and pacing, in both chambers.

According to various embodiments of the subject technology, a non-transitory machine-readable medium encoded with instructions executable by a processing system to perform methods described herein is also provided.

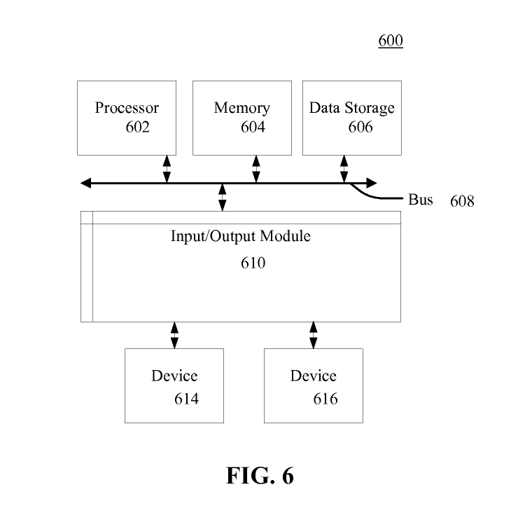

For example, FIG. 6 a block diagram illustrating an exemplary computer system 600 with which embodiments of the subject technology can be implemented. In certain embodiments, the computer system 600 may be a device for modulating the function (such as conductance) of an ion channel. Such a device, for example, can be configured to attach to a patient to deliver ACs with suitable resonant frequencies. In certain embodiments, the computer system 600 may be implemented using hardware or a combination of software and hardware, either in a dedicated server, or integrated into another entity, or distributed across multiple entities.

The computer system 600 includes a bus 608 or other communication mechanism for communicating information, and a processor 602 coupled with the bus 608 for processing information. By way of example, the computer system 600 may be implemented with one or more processors 602. The processor 602 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, and/or any other suitable entity that can perform calculations or other manipulations of information.

The computer system 600 can include, in addition to hardware, code that creates an execution environment for the computer program in question, e.g., code that constitutes processor firmware, a protocol stack, a database management system, an operating system, or a combination of one or more of them stored in an included memory 604, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, and/or any other suitable storage device, coupled to the bus 608 for storing information and instructions to be executed by the processor 602. The processor 602 and the memory 604 can be supplemented by, or incorporated in, special purpose logic circuitry.

The instructions may be stored in the memory 604 and implemented in one or more computer program products, i.e., one or more modules of computer program instructions encoded on a computer readable medium for execution by, or to control the operation of, the computer system 600, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and/or application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and/or xml-based languages. The memory 604 may also be used for storing temporary variable or other intermediate information during execution of instructions to be executed by the processor 602.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

The computer system 600 further includes a data storage device 606 such as a magnetic disk or optical disk, coupled to the bus 608 for storing information and instructions. The computer system 600 may be coupled via an input/output module 610 to various devices (e.g., devices 614 and 616). The input/output module 610 can be any input/output module. Exemplary input/output modules 610 include data ports (e.g., USB ports), audio ports, and/or video ports. In some embodiments, the input/output module 610 includes a communications module. Exemplary communications modules include networking interface cards, such as Ethernet cards, modems, and routers. In certain aspects, the input/output module 610 is configured to connect to a plurality of devices, such as an input device 614 and/or an output device 616. Exemplary input devices 614 include a keyboard and/or a pointing device (e.g., a mouse or a trackball) by which a user can provide input to the computer system 600. Other kinds of input devices 614 can be used to provide for interaction with a user as well, such as a tactile input device, visual input device, audio input device, and/or brain-computer interface device. For example, feedback provided to the user can be any form of sensory feedback (e.g., visual feedback, auditory feedback, and/or tactile feedback), and input from the user can be received in any form, including acoustic, speech, tactile, and/or brain wave input. Exemplary output devices 616 include display devices, such as a cathode ray tube (CRT) or liquid crystal display (LCD) monitor, for displaying information to the user.

According to certain embodiments, the computer system 600 can implement a method for modulating the function (such as conductance) of an ion channel in response to the processor 602 executing one or more sequences of one or more instructions contained in the memory 604. Such instructions may be read into the memory 604 from another machine-readable medium, such as the data storage device 606. Execution of the sequences of instructions contained in the memory 604 causes the processor 602 to perform the process steps described herein. One or more processors in a multi-processing arrangement may also be employed to execute the sequences of instructions contained in the memory 604. In some embodiments, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component (e.g., a data server), or that includes a middleware component (e.g., an application server), or that includes a front end component (e.g., a client computer having a graphical user interface and/or a Web browser through which a user can interact with an implementation of the subject matter described in this specification), or any combination of one or more such back end, middleware, or front end components. The components of the system 600 can be interconnected by any form or medium of digital data communication (e.g., a communication network). Examples of communication networks include a local area network and a wide area network.

The term “machine-readable storage medium” or “computer readable medium as used herein refers to any medium or media that participates in providing instructions to the processor 602 for execution. Such a medium may take many forms, including, but not limited to, non-volatile media, volatile media, and transmission media. Non-volatile media include, for example, optical or magnetic disks, such as the data storage device 606. Volatile media include dynamic memory, such as the memory 604. Transmission media include coaxial cables, copper wire, and fiber optics, including the wires that comprise the bus 608. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, a “processor” can include one or more processors, and a “module” can include one or more modules.

In an aspect of the subject technology, a machine-readable medium is a computer-readable medium encoded or stored with instructions and is a computing element, which defines structural and functional relationships between the instructions and the rest of the system, which permit the instructions' functionality to be realized. Instructions may be executable, for example, by a system or by a processor of the system. Instructions can be, for example, a computer program including code. A machine-readable medium may comprise one or more media.

As used herein, the word “module” refers to logic embodied in hardware or firmware, and/or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

Methods of generating alternating current or alternating magnetic field are well known in the art. Alternating current can be used to create a changing magnetic field, and changing magnetic fields can be used to create alternating current.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

### Exemplification

The subject technology now being generally described, it will be more readily understood by reference to the following examples, which are included merely for purposes of illustration of certain aspects and embodiments of the subject technology, and are not intended to limit the invention.

### Introduction

The Examples described herein involve the use of electrical fields oscillating at specific frequencies (individual, or combined) to modulate ion channel protein function in excitable cells.

The purpose is to provide spatially localized electrical stimuli in an organism at specific frequencies which can modulate individual or multiple ion channel proteins and thereby affect their function. Plainly speaking, the invention is a form of electro-pharmacotherapy. For example (details below), by applying sinusoidal electric fields across the heart at a frequency previously demonstrated to have some resonance with the cardiac sodium channel Nav1.5, we were able to demonstrate that we can increase cardiac conduction, consistent with a therapy that specifically targets cardiac sodium channels.

### Results and Data

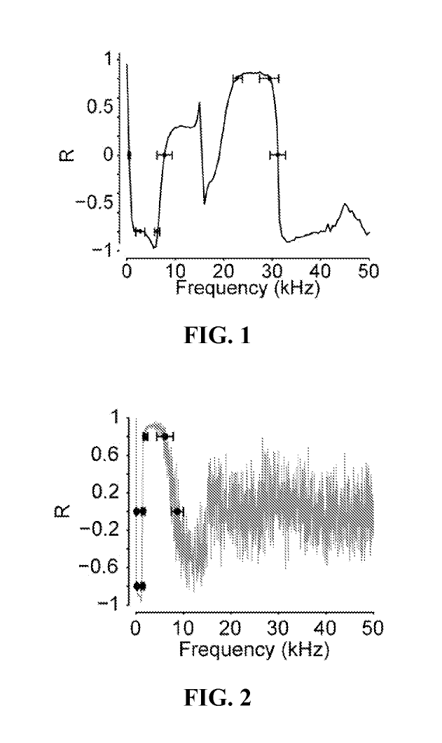

We previously demonstrated that the cardiac sodium channel (Nav1.5) and the inward rectifier potassium channel (Kir2.1) resonate at different frequencies.^{1,2} Simply, we inserted sinusoidal electric fields (henceforth referred to as “fields”) into a cell only expressing one ion channel sub-type and recorded the output current. We correlated the amplitude of fields at specific frequencies with the whole-cell peak channel conductance. FIG. 1 demonstrates which field frequencies correlate with the peak conductance of Nav1.5 and FIG. 2 demonstrates the same for Kir2.1. Importantly, these previously published figures do not demonstrate or suggest that the field affects channel conductance. They only demonstrate a correlation between which frequencies are preferentially passed or damped by an ion channel sub-type. Here, we describe the use of fields at specific frequencies for modulating the behavior of specific ion channels and thereby affect whole-heart electrophysiology for example.

Experimental Methods:

For the purpose of this example, we chose 4 field frequencies to apply across a guinea pig heart. The hearts (n=8) were isolated from guinea pigs and retrogradely perfused with an artificial blood like solution in a Langendorf preparation. Hearts were stained with the voltage sensitive dye, di-4-ANEPPS to record the electrical activity of the heart because at present, it is not possible to deliver an electric field to biological tissue and measure an electrical signal with electrodes. Stainless steel paddles of approximately 2 cm×1 cm were placed on either side of the heart (lateral positioning). The fields were delivered at a peak-to-peak amplitude of 5V for 1 minute. Conduction velocity was measured after 30 seconds of field stimulation for 2 seconds. All fields were sinusoidal, and the frequencies used in this experiment were 8.3 kHz, 25.5 kHz, 31.8 kHz and 40 kHz. The choice of frequencies was not random.
8.3 kHz fields do not correlate with Nav1.5 conductance, but positively correlate with Kir2.1 conductance
25.5 kHz fields positively correlate with Nav1.5 conductance, but not Kir2.1 conductance
31.8 kHz fields do not correlate with either Nav1.5 or Kir2.1 conductance
40 kHz fields demonstrate weak inverse correlation with Nav1.5 but no correlation with Kir2.1

Conduction velocity was measured during point stimulation of the anterior epicardial surface of the ventricles before (PRE), during (STIM), and after (POST) field stimulation. The oscillating fields were delivered in a random order for all 8 hearts.

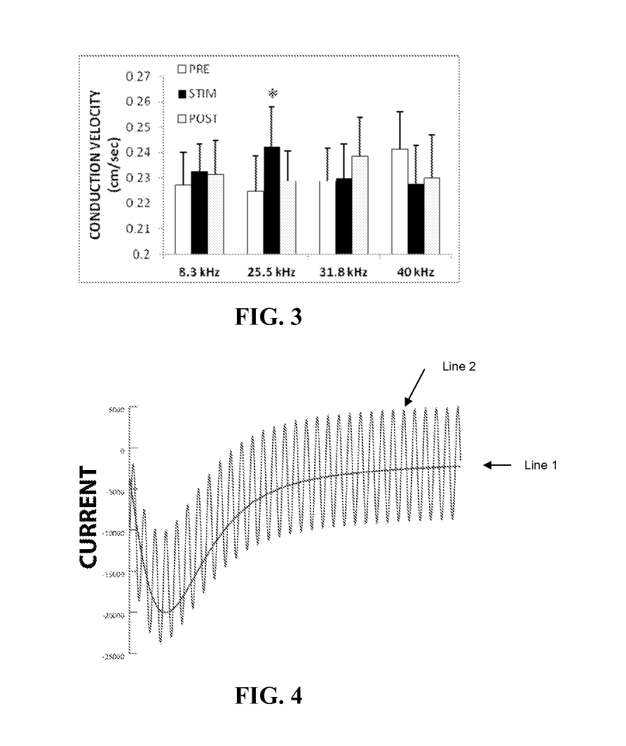

FIG. 3 contains a summary graph (n=8) of transverse conduction velocity (conduction across the short axis of cardiac myocytes). Transverse conduction is the most consistent, detailed measure of cardiac conduction. Importantly, 8.3 kHz did not change cardiac conduction, consistent with a treatment that has no effect on Nav1.5. During 25.5 kHz stimulation, cardiac conduction velocity significantly increased consistent with a treatment that increases Nav1.5 conductance. Neither 31.8 kHz or 40 kHz significantly altered cardiac conduction.

It is important to note that pre and post sinusoidal field stimulation is thought to be normal cardiac conduction and that the field acutely confers therapy. For example, the 25.5 kHz field increases conduction velocity, and upon cessation of the field (post) conduction velocity returns to pre-field levels. Interestingly, there is a non-significant increase in the post 31.8 kHz and pre-40 kHz baseline conduction velocities, both of which are not significantly different from conduction velocity during the 25.5 kHz field. It is possible that the order of field delivery affects conduction velocity after field cessation, or that certain frequencies modulate cardiac conduction during field stimulation or preferably after field stimulation.

Specificity.

As can be seen from FIGS. 1 and 2, many field frequencies demonstrate resonance in both the sodium and potassium channel. Additionally, even the 0 correlation field frequencies in a single ion channel demonstrates variability. For example, individual recordings suggest that the second 0 correlation frequency for Nav1.5 (FIG. 1) lies between approximately 6.658 and 10.084 kHz (8.371±1.713 kHz). As a result of overlap and imprecise resonant frequencies, determining frequencies with maximal effect on electrophysiology requires additional processes. Additionally, the correlation spectrum in FIGS. 1 and 2 do not indicate the magnitude of effect the resonant frequency has on a channel. For example, the current through Nav1.5 at a step potential of −40 mV is indicated by the Line 1 in FIG. 4. The same current at the same step potential but with a ±5 mV field oscillating at 25.5 kHz is represented by Line 2. It appears that the negative component of the 25.5 kHz field is more negative than the current represented by the Line 1. Yet the positive component of the same field is more positive than the current represented by the Line 1. Therefore, the observation that 25.5 kHz seems to increase cardiac conduction in a whole heart, possibly due to the early activation of the sodium channel, increased current through the channel, or some other mechanisms.

We conclude that certain field frequencies modulate cardiac conduction. These frequencies seem to correlate with some resonant frequencies of ion channels that could modulate cardiac conduction (Nav1.5).

In various embodiments, oscillating voltages may be applied to the heart at other frequencies. For example, the oscillating voltage may be applied at frequencies of at least 1 kHz, at least 2 kHz, at least 3 kHz, at least 4 kHz, at least 5 kHz, at least 10 kHz, at least 15 kHz, at least 20 kHz, at least 25 kHz, at least 30 kHz, at least 35 kHz, at least 40 kHz, at least 45 kHz, or at least 50 kHz. In certain embodiments, the oscillating voltage may be applied at frequencies of at most 1 kHz, at most 2 kHz, at most 3 kHz, at most 4 kHz, at most 5 kHz, at most 10 kHz, at most 15 kHz, at most 20 kHz, at most 25 kHz, at most 30 kHz, at most 35 kHz, at most 40 kHz, at most 45 kHz, or at most 50 kHz. The oscillating voltage may be applied at a single frequency or within a range of frequencies.

The oscillating voltage may be applied for a defined period of time and may be repeated at regular or irregular intervals. The intervals may be repeated at intervals of at least 1 sec, at least 2 sec, at least 3 sec, at least 4 sec, at least 5 sec, at least 10 sec, at least 20 sec, at least 30 sec, at least 40 sec, at least 50 sec, at least 1 minute, at least 2 minutes, at least 3 minutes, at least 4 minutes, at least 5 minutes, at least 10 minutes, at least 20 minutes, at least 30 minutes, or at least 1 hour. The intervals may be repeated at intervals of at most 1 sec, at most 2 sec, at most 3 sec, at most 4 sec, at most 5 sec, at most 10 sec, at most 20 sec, at most 30 sec, at most 40 sec, at most 50 sec, at most 1 minute, at most 2 minutes, at most 3 minutes, at most 4 minutes, at most 5 minutes, at most 10 minutes, at most 20 minutes, at most 30 minutes, or at most 1 hour.

In various embodiments, the oscillating voltage may have a peak to peak voltage of at least 1 mV, at least 2 mV, at least 3 mV, at least 4 mV, at least 5 mV, at least 10 mV, at least 15 mV, at least 20 mV, at least 25 mV, at least 30 mV, at least 40 mV, at least 50 mV, at least 60 mV, at least 70 mV, at least 80 mV, at least 90 mV, at least 100 mV, at least 200 mV, at least 300 mV, at least 400 mV, at least 500 mV, at least 750 mV, at least 1 V, at least 2 V, at least 3 V, at least 4 V, or at least 5 V. In certain embodiments, the oscillating voltage may have a peak to peak voltage of at most 1 mV, at most 2 mV, at most 3 mV, at most 4 mV, at most 5 mV, at most 10 mV, at most 15 mV, at most 20 mV, at most 25 mV, at most 30 mV, at most 40 mV, at most 50 mV, at most 60 mV, at most 70 mV, at most 80 mV, at most 90 mV, at most 100 mV, at most 200 mV, at most 300 mV, at most 400 mV, at most 500 mV, at most 750 mV, at most 1 V, at most 2 V, at most 3 V, at most 4 V, or at most 5 V. The oscillating voltage in various embodiments may be superimposed on a DC voltage of about 1 mV, about 2 mV, about 3 mV, about 4 mV, about 5 mV, about 10 mV, about 15 mV, about 20 mV, about 25 mV, about 30 mV, about 40 mV, about 50 mV, about 60 mV, about 70 mV, about 80 mV, about 90 mV, about 100 mV, about 200 mV, about 300 mV, about 400 mV, about 500 mV, about 750 mV, about 1 V, about 2 V, about 3 V, about 4 V, or about 5 V, about 0V, about −1 mV, about −2 mV, about −3 mV, about −4 mV, about −5 mV, about −10 mV, about −15 mV, about −20 mV, about −25 mV, about −30 mV, about −40 mV, about −50 mV, about −60 mV, about −70 mV, about −80 mV, about −90 mV, about −100 mV, about −200 mV, about −300 mV, about −400 mV, about −500 mV, about −750 mV, about −1 V, about −2 V, about −3 V, about −4 V, or about −5 V.

The particular frequency, peak to peak voltage, DC voltage, and timing characteristics may be chosen to modulate one or more sodium channel type and/or one or more potassium type, where modulation includes increasing or decreasing conductance through the particular channel type(s). Modulation using oscillating voltage application may treat one or more cardiac condition as discussed herein.

Process for Determining Efficacy.

In this example, we propose a few processes for identifying efficacious field frequencies.

Using the protocol outlined in the manuscripts by Rigby and Poelzing,^{1,2} it is possible to use white noise in heterologous expression systems to create a map of resonant field frequencies for any ion channel. This process can be expanded to ion channels with the alpha subunit and accessory protein subunits expressed in heterologous expression systems. Once the resonant frequencies have been identified, as in FIGS. 1 and 2, any field frequency with high positive or negative correlation with peak channel conductance can be used as first values for exploring field frequency efficacy.

This process can be entirely bypassed by randomly choosing frequencies and using any type of artificial intelligence algorithm such as, but not limited to, a genetic search heuristic algorithm. Search heuristics could also use identified resonant frequencies as seed or training values. The parameters to be searched would be:

1. Field frequency

2. Field amplitude

3. Field orientation.

The output would be a quantifiable biological parameter. In the example above, the output would be cardiac conduction velocity. Outputs could though include action potential morphology or action potential duration as additional examples.

### Discussions

Several drugs are available to inhibit or increase function of specific ion channels. However, these drugs have two shortcomings First, drugs are usually delivered systemically, which means they can interact with any target ion channel throughout the body. Drugs usually have non-specific interactions and are difficult to deliver only to the target site. Limitations in drug delivery systems make it difficult to administer a constant drug dose to the target area. Second, drugs are usually delivered as a bolus or by a pump, which means they cannot be turned on and off quickly. Typically, drug treatment is highest at the time of delivery, and decays thereafter. The technology exemplified herein can be delivered to a target organ such as the heart without interacting with the brain for example. It can be turned on and off to acutely correct a physiologic abnormality.

Electropharmaco Therapy.

We describe the approach of current methods of electro-pharmacotherapy applied to the heart through pace-makers or cardiac defibrillators. Modern application of electro-pharmacotherapy seeks to grossly reset systems or entrain systems such that when the electro-pharmacotherapy is released, excitable cells can presumably return to some resting state ready for some normal electrical excitatory pathway. There are two main limitations with this approach as described above. Therefore, a more direct approach with lower power requirements might be to specifically modulate ion channels responsible for intracellular calcium accumulation in real-time.

Even the work by Tandri et al.,³ which uses sinusoidal waves to defibrillate hearts provides therapy after the arrhythmia has begun, but cannot prevent arrhythmias. Until now, no one has suggested tuning the frequency to achieve specific and different results. The technology described herein may be used to prevent development of pro-arrhythmic states from developing and is different from the technology described by Tandri et al.

In summary, we previously demonstrated that two cardiac ion channels exhibit distinct frequency responses to externally applied oscillating electrical fields with amplitudes between 25 m V and 100 m V. Subsequently, we identified frequencies with high positive, negative and no correlation with channel conductance. As a reduction to practice, we applied a frequency with high positive correlation to an intact guinea pig heart. We discovered that the application of one frequency whose amplitude positively correlates with the cardiac sodium channel, increases cardiac conduction demonstrating that an AC current can acutely modulate cardiac electrophysiology without providing sufficient current to excite cells by canonical mechanisms.

We've demonstrated that oscillating fields can be used to modulate cardiac electrophysiology. The technology is applicable to any excitable cell. This means the technology has implications for cancer, heart disease, hearing loss, neurological disorders such as Alzheimer's, Parkinsons, or epilepsy. More technically, oscillating fields can modulate the function of any biological cell and can be used as an electrical therapy for acutely and focally treating disorders associated with protein dysfunction.

Current cardiac Electropharmacotherapy relies on pacing or applying large potentially damaging electrical stimuli to tissue in order to bluntly reset or resynchronize electrical activity. Such technologies rely on indiscriminately activating or inhibiting all cells within a region without specificity to particular protein function. Our technology uses signals tuned to frequencies that elicit reactions in specific proteins. The technology uses less power, causing less damage while still allowing for treatment in specific areas of the body. Furthermore, the treatment is instantaneous.

This technology is useful to all drug delivery fields where a protein has been identified as the putative mechanism of the pathophysiology. For example, rather than administering a cardiac specific drug with some non-specificity and time course to peak action, an electrical device could be used to monitor and correct cardiac electrophysiology in real time. Similarly, it can be used prophylactically to prevent lethal electrical abnormalities and spare the patient the life-restoring and often painful use of defibrillation or deep brain stimulation. This technology can also be used for greater specificity and spatial control during functional electrical stimulation to control limbs or prosthetics.

### REFERENCES

1. Rigby J R, Poelzing S. Recreation of an Ion Channel Current IV Curve Using Frequency Components. J Vis Exp. 2011 Feb. 8; (48)
2. Rigby J R, Poelzing S. A Novel Frequency Analysis Method for Assessing K(ir)2.1 and Na (v)1.5 Currents. Ann Biomed Eng. 2012 April; 40(4):946-54
3. Tandri H, Weinberg S H, Chang K C, Zhu R, Trayanova N A, Tung L, Berger R D, Reversible cardiac conduction block and defibrillation with high-frequency electric field. Sci Transl Med. 2011 Sep. 28; 3(102):102ra96.
4. Tanner, Reversible blocking of nerve conduction by alternating-current excitation. Nature 195, 712-713 (1962).

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents and NCBI Entrez or gene ID sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A method of modulating the conductance of an ion channel of an excitable cell, comprising: applying an alternating current (AC) at a frequency that resonates with said ion channel.

2. The method of claim 1, wherein said excitable cell is a cardiomyocyte.

3. The method of claim 1, wherein said ion channel is a Na⁺ Channel.

4. The method of claim 3, wherein said Na+ Channel is Naᵥ1.5.

5. The method of claim 4, wherein said frequency is about 25.5 kHz.

6. The method of claim 4, wherein said frequency is about 40 kHz.

7. The method of claim 1, wherein said ion channel is a K⁺ Channel.

8. The method of claim 7, wherein said K⁺ Channel is Kᵢᵣ2.1.

9. The method of claim 8, wherein said frequency is about 31.8 kHz.

10. A method of treating dysrhythmia in a subject in need thereof, comprising applying to said subject an alternating current (AC) at a frequency that resonates with an ion channel of a cardiomyocyte.

11. The method of claim 10, wherein said ion channel is a Na⁺ Channel.

12. The method of claim 11, wherein said Na⁺ Channel is Naᵥ1.5.

13. The method of claim 11, wherein said frequency is about 25.5 kHz.

14. The method of claim 11, wherein said frequency is about 40 kHz.

15. The method of claim 10, wherein said ion channel is a K⁺ Channel.

16. The method of claim 15, wherein said K⁺ Channel is Kᵢᵣ2.1.

17. The method of claim 16, wherein said frequency is about 31.8 kHz.

18. A device for treating dysrhythmia, comprising: a computer or microprocessor-readable program containing one or more algorithms for generating or delivering alternating current (AC); a plurality of electrodes; and a waveform generator; wherein the device is configured to generate an alternating current (AC) at a frequency that resonates with an ion channel of a cardiomyocyte.

19. The device of claim 18, wherein said ion channel is a Na⁺ Channel.

20. The device of claim 19, wherein said Na⁺ Channel is Naᵥ1.5.

21. The device of claim 19, wherein said frequency is about 25.5 kHz.

22. The device of claim 19, wherein said frequency is about 40 kHz.

23. The device of claim 18, wherein said ion channel is a K⁺ Channel.

24. The device of claim 23, wherein said K⁺ Channel is Kᵢᵣ2.1.

25. The method of claim 24, wherein said frequency is about 31.8 kHz.
